(19) United States
(12) Patent Application Publication
(10) Pub. No.: US 2025/0082279 A1
(43) Pub. Date: Mar. 13, 2025

(54) iNPH PREDICTION METHOD BASED ON THE VISUAL ODDBALL PARADIGM

(71) Applicant: TIANJIN UNIVERSITY, Tianjin City (CN)

(72) Inventors: Dong MING, Tianjin City (CN); Xiuyun LIU, Tianjin City (CN); Zihua ZHANG, Tianjin City (CN); Huijie YU, Tianjin City (CN); Congying HAO, Tianjin City (CN); Kai YU, Tianjin City (CN); Xiaoyi WANG, Tianjin City (CN); Fang GUO, Tianjin City (CN); Zaiyu GUO, Tianjin City (CN); Yan XING, Tianjin City (CN); Yulin SUN, Tianjin City (CN)

(73) Assignee: TIANJIN UNIVERSITY, Tianjin City (CN)

(21) Appl. No.: 18/407,501

(22) Filed: Jan. 9, 2024

(30) Foreign Application Priority Data
Sep. 11, 2023 (CN) 2023111679142

(51) Int. Cl. A61B 5/00; A61B 5/291; A61B 5/378; G16H 50/30

(52) U.S. Cl. CPC A61B 5/7275; A61B 5/291; A61B 5/378; A61B 5/4088; G16H 50/30

(57) ABSTRACT

The present invention relates to the technical field of medical data processing, and discloses an iNPH prediction method based on a visual Oddball paradigm, comprising: performing the same visual Oddball paradigm experiments on a target population before and after the target population undergoes LTT, and obtaining an electroencephalography (EEG) signal data; Pre-processing the EEG signal data, and obtaining an event-related potential feature, the event-related potential feature being a P300 amplitude feature; Based on the event-related potential feature, the iNPH prediction model is trained to obtain the iNPH prediction model. The present invention can obtain more objective EEG data through the visual Oddball paradigm experiment, analyze the characteristic change of the P300 amplitude feature of the EEG data, and quantify the degree of improvement of cognitive function, and train to obtain the iNPH prediction model, which can be used to assist the doctor to quickly and accurately diagnose the iNPH, so as to enable the patient to receive timely and effective treatment, which can reduce the burden of medical care and bring considerable medical value.

### TECHNICAL FIELD

The present invention relates to the technical field of medical data processing, and in particular to an iNPH prediction method based on the visual Oddball paradigm.

### BACKGROUND ART

Idiopathic Normal Pressure Hydrocephalus refers to the syndrome of enlarged cerebral ventricles with normal cerebrospinal fluid (CSF) pressure on the image, which is characterized by unsteady gait, dementia, and urinary incontinence, and excludes the secondary factors of hemorrhage, trauma, meningitis, and intracranial occupancy. iNPH is more common among elderly people, and severely affects the normal life of the patients. Patients with iNPH can improve the above symptoms through cerebrospinal fluid shunt surgery, thus reducing the burden on society and family. However, the three main symptoms of iNPH are also commonly found in other geriatric diseases such as Alzheimer's Disease (AD), which often cause family members and even doctors to misinterpret the above manifestations of iNPH as the result of the patient's aging, which makes the diagnosis and treatment of iNPH patients more difficult. Lumbar Tap Test (LTT) is a common clinical method to distinguish iNPH from other diseases. Before and after LTT, doctors observe the degree of improvement in cognitive function and gait of patients suspected of having iNPH. When a patient suspected of having iNPH shows significant improvement in cognitive function, gait and urinary dysfunction after LTT, the patient is clinically diagnosed as having iNPH.

The Chinese Guidelines for the Clinical Management of Idiopathic Normal Pressure Hydrocephalus 2022 states that the commonly used methods for evaluating cognitive function in clinical practice generally use the Mini-Mental State Examination (MMSE) scale and the Montreal Cognitive Assessment (MoCA) scale. However, the assessment results based on the scale are easily affected by the subjective factors of patients, especially their education level, personal life experience and learning effects, which leads to lower accuracy of the assessment results.

Therefore, there is an urgent need for a method that can assist physicians in accurately diagnosing iNPH.

### SUMMARY

The present invention provides an iNPH prediction method based on the visual Oddball paradigm to address the shortcoming that the prior art is unable to assist physicians in accurately diagnosing iNPH.

The present invention provides a method for constructing an iNPH prediction model based on the visual Oddball paradigm, comprising:

The visual Oddball paradigm experiment was conducted on the target population before lumbar puncture and LTT, and the data of standard stimulation EEG and target stimulation EEG before LTT were obtained

After lumbar puncture and drainage, the same visual Oddball paradigm experiment was performed on the target population to obtain the standard stimulation EEG data and target stimulation EEG data after LTT.

Standard stimulation EEG data before LTT, target stimulation EEG data before LTT, standard stimulation EEG data after LTT and target stimulation EEG data after LTT were preprocessed. The characteristics of event-related potentials to standard stimulus events before LTT, event-related potentials to target stimulus events before LTT, event-related potentials to standard stimulus events after LTT and event-related potentials to target stimulus events after LTT were obtained, in which event-related potential features were P300 amplitude features.

The iNPH prediction model was trained according to the characteristics of event-related potentials to standard stimulus events before LTT, event-related potentials to target stimulus events before LTT, event-related potentials to standard stimulus events after LTT, and event-related potentials to target stimulus events after LTT.

One of the target populations is iNPH patients.

A method of constructing an iNPH prediction model based on a visual Oddball paradigm according to the present invention, said performing a visual Oddball paradigm experiment on a target population comprising:
Configure EEG caps for the target population;
A number of images were presented to the target population at predetermined time intervals, where stimuli with a high probability of image occurrence were defined as standard stimuli and stimuli with a low probability of image occurrence were defined as target stimuli in a visual Oddball paradigm experiment, and the target population was asked to silently memorize the number of times that the target stimuli appeared;
While presenting the images to the target population, the EEG signal data of the target population is obtained through the EEG cap.

A method of constructing an iNPH prediction model based on a visual Oddball paradigm according to the present invention, said presenting a number of images to a target population at predetermined time intervals, comprising:
A plurality of rounds of image stimuli are presented to the target population, each round of image stimuli comprising a first patterned image stimulus and a second patterned image stimulus, wherein the probability of the first patterned image appearing is 80% and the first patterned image stimulus is a standard stimulus, the probability of the second patterned image appearing is 20% and the second patterned image stimulus is a target stimulus, and wherein there is a predetermined resting time between each round of image stimuli;
The first pattern image or the second pattern image is presented to the target population each time in accordance with a predetermined maintenance duration, and there is a predetermined time interval between each pattern image stimulation.
A method of constructing an iNPH prediction model based on a visual Oddball paradigm according to the present invention, said configuring an EEG cap for a target population, specifically:
The target population was equipped with EEG caps, which used standard Ag/AgCl electrodes, with electrodes placed with reference to the international 10-20 system, and the number of channels was 32 leads, the parameters of the EEG caps were set to a sampling rate of 1000 Hz and a band-pass filtering of 0.1 to 200 Hz, and the acquisition process was performed with the top of the head of the target population as a reference, with the forehead grounded and the impedance between the scalp and the electrodes kept below 10 KΩ.

A method of constructing an iNPH prediction model based on a visual Oddball paradigm according to the present invention, said preprocessing comprising: filtering out invalid data, filtering, downsampling, data segmentation, and baseline correction, wherein.

Filtering out invalid data is specified as checking for bad lead data in the EEG signal data and filtering it out;
Filtering is specified as the EEG signal data is filtered using a filter with a filter range of 0.5 to 30 Hz;
Downsampling is specified as downsampling the EEG signal data to 200 Hz;
The data segmentation is specified as the start moment of each image stimulation is defined as the zero moment, and the EEG signal data is segmented and intercepted in accordance with a first predetermined time window;
The baseline correction is specified as baseline correcting the EEG signal data obtained from each image stimulation by using the EEG signal data segment of the second predetermined time window as a baseline.

According to the construction method of iNPH prediction model based on visual Oddball paradigm provided by the invention, the standard stimulation EEG data before LTT, the target stimulation EEG data before LTT, the standard stimulation EEG data after LTT and the target stimulation EEG data after LTT are preprocessed. The characteristics of event-related potentials to standard stimulus events before LTT, event-related potentials to target stimulus events before LTT, event-related potentials to standard stimulus events after LTT and event-related potentials to target stimulus events after LTT were obtained, including:

Based on the preprocessed EEG signal data, the event-related potential characteristics were obtained by averaging the EEG signal data of each trial separately at each electrode by overlaying the calculation.

According to a method for constructing an iNPH prediction model based on a visual Oddball paradigm provided by the present invention, the event-related potential features of the healthy population and the iNPH patient have different forms of expression, wherein the event-related potential features of the iNPH patient have the following specific forms of expression: a standard stimulus event-related potential feature before LTT, a target stimulus event-related potential feature before LTT, and a target stimulus event-related potential feature after LTT The standard stimulus event-related potential feature and after LTT target stimulus event-related potential feature reflect that the cognitive function of iNPH patients has been improved, but has not reached the minimum cognitive function level of the healthy population;

Wherein, said iNPH prediction model is trained to obtain the iNPH prediction model based on the before LTT standard stimulus event-related potential feature, the before LTT target stimulus event-related potential feature, the after LTT standard stimulus event-related potential feature, and the after LTT target stimulus event-related potential feature, as follows:

The iNPH prediction model was trained to obtain the iNPH prediction model based on the change characteristics of the standard stimulus event-related potential feature before LTT, the target stimulus event-related potential feature before LTT, the standard stimulus event-related potential feature after LTT, and the target stimulus event-related potential feature after LTT.

The present invention also provides an apparatus for constructing an iNPH prediction model based on the visual Oddball paradigm, comprising:
The data acquisition module is configured to: The target population received the standard stimulation EEG data and target stimulation EEG data obtained by visual Oddball paradigm experiment before LTT. The standard stimulation EEG data and target stimulation EEG data were obtained from the same visual Oddball paradigm experiment after LTT.

The data processing module is configured to: The data acquisition module preprocesses the standard stimulation EEG data before LTT, the target stimulation EEG data before LTT, the standard stimulation EEG data after LTT and the target stimulation EEG data after LTT. The characteristics of potential related to standard stimulus events before LTT, potential related to target stimulus events before LTT, potential related to standard stimulus events after LTT and potential related to target stimulus events after LTT were obtained, in which event-related potential features were P300 amplitude features.

The model training module is configured to train the iNPH prediction model according to the event related potential features of the standard stimulus event before LTT, the event related potential features of the target stimulus before LTT, the event related potential features of the standard stimulus after LTT, and the event related potential features of the target stimulus after LTT.

One of the target populations is iNPH patients.

The present invention also provides an iNPH prediction device based on the visual Oddball paradigm, comprising:

The data acquisition module is configured to: The standard stimulation EEG data and target stimulation EEG data obtained by visual Oddball paradigm experiment before LTT were obtained. The standard stimulation EEG data and target stimulation EEG data were obtained by the same visual Oddball paradigm experiment after LTT.

The iNPH prediction module is configured to: The iNPH prediction model is obtained by inputting the before LTT standard stimulation EEG data, before LTT target stimulation EEG data, after LTT standard stimulation EEG data and after LTT target stimulation EEG data obtained by the data acquisition module into any of the above iNPH prediction model construction methods based on visual Oddball paradigm to obtain the iNPH prediction results;
wherein said tester is a patient with suspected iNPH, a patient with iNPH to be ruled out, or a patient with a triad of manifestations of gait disturbance, urinary incontinence, and cognitive impairment.

The present invention also provides a brain-computer interface system for predicting iNPH, comprising:
An EEG cap for being worn on the head of a tester to obtain EEG signals from said tester;
An EEG acquisition device configured to be coupled to the electrodes of said EEG cap to acquire EEG signals of said tester acquired by said EEG cap;
A computer comprising a communication module, a memory, a processor, and a computer program stored on said memory and runnable on said processor, wherein said computer is configured to communicate with said electroencephalographic acquisition device to obtain an electroencephalographic signal from said tester, and wherein said processor is configured to implement the following steps in executing said computer program:
Obtain before LTT standard stimulus EEG signal data and before LTT target stimulus EEG signal data obtained from a visual Oddball paradigm experiment before the tester underwent a lumbar puncture drain, and obtain after LTT standard stimulus EEG signal data and after LTT target stimulus EEG signal data obtained from the same visual Oddball paradigm experiment after the tester underwent a lumbar puncture drain;
Preprocessing was performed on the before LTT standard stimulation EEG signal data, before LTT target stimulation EEG signal data, after LTT standard stimulation EEG signal data and after LTT target stimulation EEG signal data;
The preprocessed before LTT standard stimulation EEG signal data, before LTT target stimulation EEG signal data, after LTT standard stimulation EEG signal data and after LTT target stimulation EEG signal data are inputted into the iNPH prediction model obtained by the method of constructing an iNPH prediction model based on the visual Oddball paradigm described in any of the foregoing claims, to obtain the prediction result of whether the tester is an iNPH patient or not.
wherein said tester is a patient with suspected iNPH, a patient with iNPH to be ruled out, or a patient with a triad of manifestations of gait disturbance, urinary incontinence, and cognitive impairment.

The present invention provides an iNPH prediction method based on the visual Oddball paradigm, and designs a visual Oddball paradigm experiment that is not easily affected by subjective factors such as the patient's cultural level and life experience, and the target population can obtain more objective EEG data by performing the visual Oddball paradigm experiment before and after LTT, and the event-related potential feature (P300 amplitude feature) characteristic change of the EEG data can be quantified by analyzing the event-related potential feature of the EEG data. By analyzing the event-related potential features (P300 amplitude features) of the EEG data, the degree of improvement of cognitive function can be quantified, and the iNPH prediction model obtained from this training can be used to assist doctors to make iNPH diagnosis quickly and accurately, so that patients can receive effective treatment in time, which can reduce the burden of medical care and bring considerable medical value.

### BRIEF DESCRIPTION OF THE DRAWINGS

In order to more clearly illustrate the technical solutions in the present invention or prior art, the following will make a brief introduction to the accompanying drawings that need to be used in the description of the embodiments or prior art, and it will be obvious that the accompanying drawings in the following description are some of the embodiments of the present invention, and for the person of ordinary skill in the field, other accompanying drawings can be obtained based on these drawings without putting in creative labor.

FIG. 1 shows a flowchart of a method for constructing an iNPH prediction model based on a visual Oddball paradigm provided by the present invention.

FIG. 2 illustrates the 10-20 international standard electrode distribution.

FIG. 3 illustrates an example visual Oddball paradigm experiment.

FIG. 4 illustrates the time-domain waveforms of a healthy population under the visual Oddball paradigm experiment.

FIGS. 5 (a)-(c) illustrate the time-domain waveforms of the iNPH population under the visual Oddball paradigm experiment.

FIG. 6 shows a schematic structure of an apparatus for constructing an iNPH prediction model based on the visual Oddball paradigm provided by the present invention.

FIG. 7 shows a schematic diagram of the structure of an electronic device provided by the present invention.

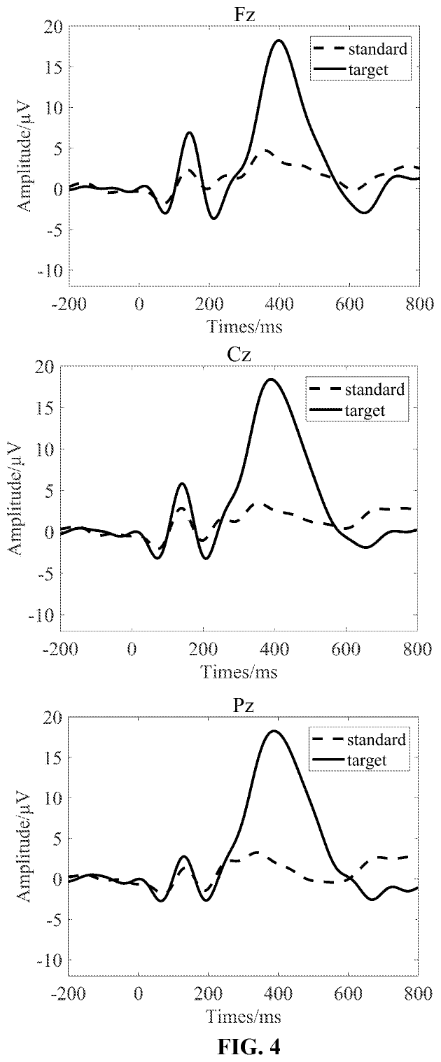

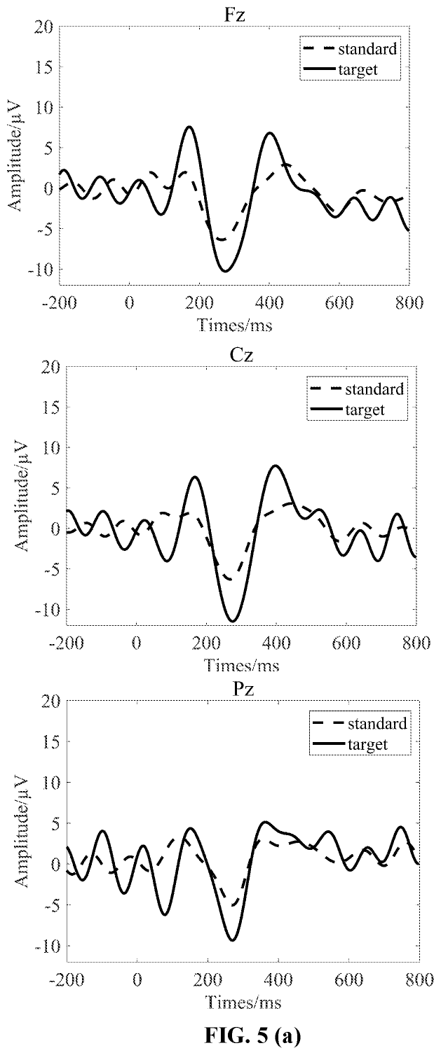

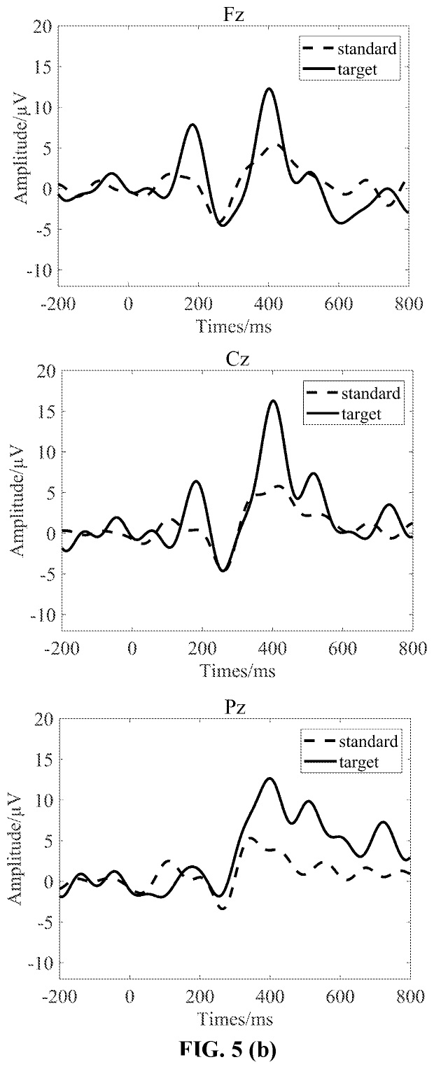

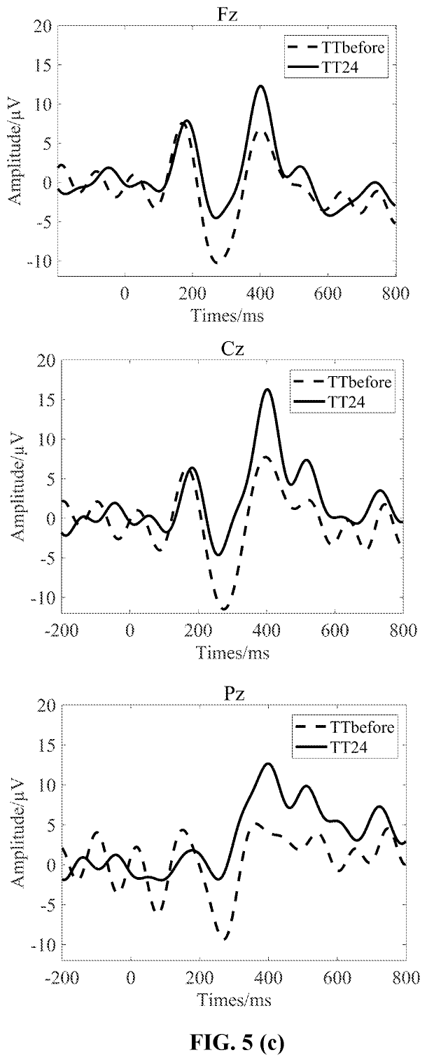

Where, in FIG. 4 to FIG. 5(c), time/ms denotes time/milliseconds, amplitude denotes amplitude, Fz, Cz, and Pz denote midline electrodes, standard denotes standard stimulus, target denotes target stimulus, TTbefore denotes before LTT, and TT24 denotes 24 h (hours) after LTT.

### DETAILED DESCRIPTION OF THE EMBODIMENTS

In order to make the objects, technical solutions and advantages of the present invention clearer, the technical solutions in the present invention will be described clearly and completely in the following in conjunction with the accompanying drawings in the present invention, and it is obvious that the described embodiments are part of the embodiments of the present invention and not all of them, and they should not be construed as limitations of the present invention. Based on the embodiments in the present invention, all other embodiments obtained by a person of ordinary skill in the art without making creative labor are within the scope of protection of the present invention. In the description of the present invention, it is to be understood that the terms used are used for descriptive purposes only and are not to be construed as indicating or implying relative importance.

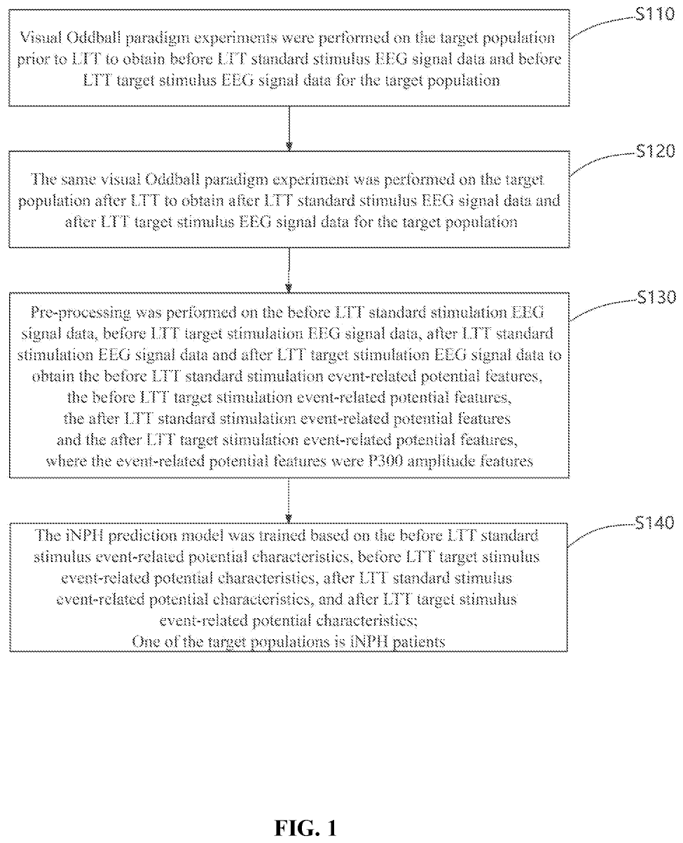

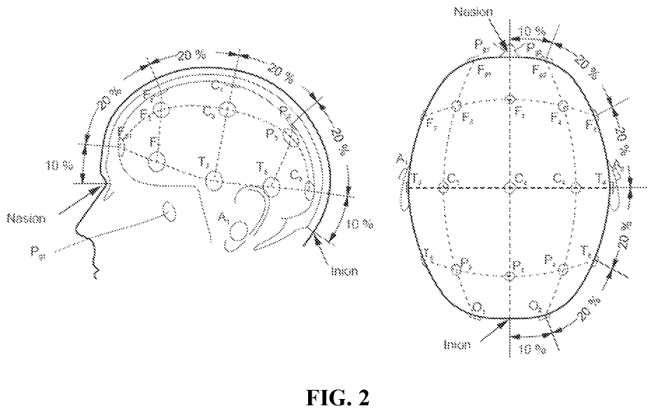

An iNPH prediction method based on the visual Oddball paradigm provided by the present invention is described below in conjunction with FIG. 1-7.

FIG. 1 is a flow diagram of a construction method of an iNPH prediction model based on a visual Oddball paradigm provided by the present invention. Referring to FIG. 1, a method for constructing an iNPH prediction model based on a visual Oddball paradigm provided by the present invention may comprise:
Step S110: Before the target population is subjected to LTT, a visual Oddball paradigm experiment is performed on the target population to obtain before LTT standard stimulation EEG signal data and before LTT target stimulation EEG signal data for the target population;
Step S120: After the target population is subjected to LTT, the same visual Oddball paradigm experiment is performed on the target population to obtain after LTT standard stimulus EEG signal data and after LTT target stimulus EEG signal data for the target population;
Step S130: The pre-processing of the before LTT standard stimulation EEG signal data, the before LTT target stimulation EEG signal data, the after LTT standard stimulation EEG signal data, and the after LTT target stimulation EEG signal data is performed to obtain the before LTT standard stimulation Event-Related Potential feature, the before LTT target stimulation Event-Related Potential feature, the after LTT standard stimulation Event-Related Potential feature, and the after LTT target stimulation Event-Related potential features (Event-Related Potentials features, ERP features), where the event-related potential feature is the P300 amplitude feature, which refers to the amplitude feature near the 300th millisecond after the stimulus occurred;
Step S140: The iNPH prediction model is trained to obtain the iNPH prediction model based on the standard stimulus event-related potential feature before LTT, the target stimulus event-related potential feature before LTT, the standard stimulus event-related potential feature after LTT, and the target stimulus event-related potential feature after LTT.

It should be noted that the target population is iNPH patients.

It should be noted that the Oddball paradigm refers to experiments in which two stimuli acting on the same sensory channel (e.g., vision) are presented randomly, where the stimulus with a high probability of occurrence is referred to as the criterion stimulus, and the stimulus with a low probability of occurrence is referred to as the target stimulus. The Oddball paradigm experiments are specifically designed to evoke the P300 amplitude feature.

It should be noted that Brain Computer Interface (BCI) is a technique for direct communication between the human brain and an output device, and Electroencephalography (EEG) is currently a more commonly used method for BCI due to its advantages of being non-invasive, easy to use, and low cost. This application is based on the technical realization of BCI based on EEG.

In one embodiment, said visual Oddball paradigm experiment on a target population comprises:
Configure EEG caps for the target population;
A number of images were presented to the target population at preset time intervals, where stimuli with a high probability of image occurrence were defined as standard stimuli and stimuli with a low probability of image occurrence were defined as target stimuli in a visual Oddball paradigm experiment, and the target population was asked to silently memorize the number of times the target stimuli appeared and ultimately provide feedback on the number of times;
While presenting the images to the target population, the EEG signal data of the target population is obtained through the EEG cap.

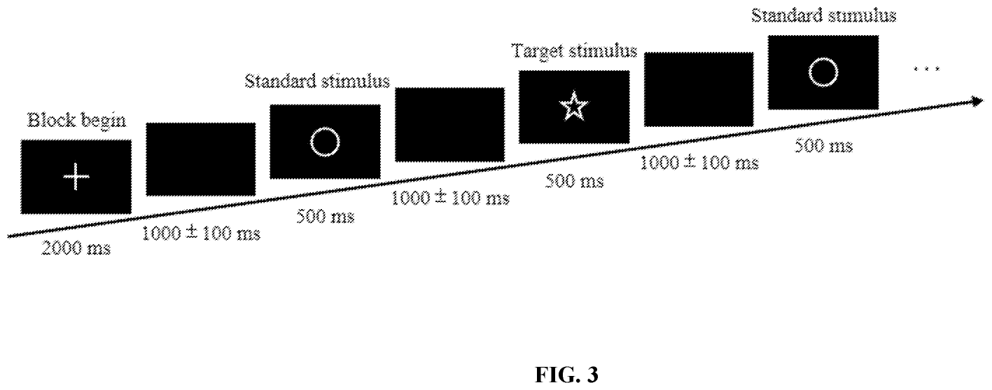

Specifically, with reference to FIG. 2, an EEG cap is configured for the target population, the EEG cap uses standard Ag/AgCl electrodes, the electrodes are placed with reference to the international 10-20 system, the number of channels is 32 leads, the parameters of the EEG cap are set to a sampling rate of 1,000 Hz and a bandpass filtering of 0.1-200 Hz, while a 50 Hz trap is used to filter out the interference of the work frequency, and the acquisition process is based on the top of the head of the target population, with the top of the head of the target population being used as a reference. The forehead was grounded, and the impedance between the scalp and the electrode was kept below 10 KΩ. During the experiment, the target population was asked to remain as still as possible, avoiding random eye movements and subtle movements not related to the task, in order to ensure the reliability of the collected EEG data.

Specifically, the SynAmps2 EEG amplifier can be electrically connected to the EEG cap to obtain EEG signal data.

In one embodiment, said presenting a number of images to a target population at predetermined time intervals comprises:
A plurality of rounds of image stimuli are presented to the target population, each round of image stimuli comprising a first patterned image stimulus and a second patterned image stimulus, wherein the probability of the first patterned image appearing is 80% and the first patterned image stimulus is a standard stimulus, the probability of the second patterned image appearing is 20% and the second patterned image stimulus is a target stimulus, and wherein there is a predetermined resting time between each round of image stimuli;
The first pattern image or the second pattern image is presented to the target population each time in accordance with a predetermined maintenance duration, and there is a predetermined time interval between each pattern image stimulation.

Specifically, in this embodiment, the same visual Oddball paradigm experiment is performed on the target population once around the same time before and after LTT (e.g., 24 hours before and after the LTT), and each time comprises two rounds of image stimulation, two blocks, and there can be a rest of 5˜10 minutes (preset rest time) between the two blocks depending on the state of the target population, and the experiment can be performed by means of monitor can be used to present images to the target population, and the resolution of the monitor can be 1920×1080 with a refresh rate of 60 Hz.

The visual Oddball paradigm used in this embodiment is shown in FIG. 3, with a circular pattern image as the first pattern image and a pentagram pattern image as the second pattern image, and each block contained 150 randomly presented trials, of which 120 trials (80%) were standard stimuli and 30 trials (20%) were target stimuli. The preset maintenance duration of each trial was 500 ms, and the preset time interval was 1500±100 ms. Before the start of the experiment, the target population was asked to silently memorize the number of times the target stimulus appeared and eventually feedback the number of times information to maintain their focus on the experiment, and the results of the experiment proved that because of the lower level of cognitive functioning of the iNPH, the correctness of the feedback of the number of times the target stimulus appeared in the iNPH was much smaller than that in the healthy population.

In one embodiment, said preprocessing comprises: filtering out invalid data, filtering, downsampling, data segmentation, and baseline correction.

Specifically, the before LTT standard stimulation EEG signal data, the before LTT target stimulation EEG signal data, the after LTT standard stimulation EEG signal data, and the after LTT target stimulation EEG signal data can be preprocessed by the EEGLAB open source toolkit to ensure the accuracy of the data.

Filtering invalid data is as follows: EEG data are imported into the EEGLAB toolkit for data preview, and information such as the number of leads, the total length of the data, the number of epochs of the data, the number of events contained in the data, and the data sampling rate of the data can be observed, and the data preview is used for the preliminary understanding and examination of the data, so as to excavate the bad leads or other invalid data (i.e., information that is obviously inconsistent with the experimental design) from EEG data and filter out the invalid data.

The filtering is specific: the EEG signal is weak and susceptible to various noise interferences, including physiological noises such as heartbeat and EMG, as well as external factors such as acquisition equipment. Since this application mainly studies the P300 amplitude characteristics, this embodiment uses a third order Butterworth filter with a filter range of 0.5-30 Hz to filter the EEG signal data.

Downsampling is specific: generally commonly used EEG signal data is mainly concentrated in 100 Hz or less, in order to improve the computing efficiency, under the premise of ensuring that the signal is not distorted, this embodiment downsamples the EEG signal data to 200 Hz.

The data segmentation is specified as follows: the EEG signal data obtained from the acquisition includes the entire experimental process, while the present application actually uses the response signal generated after the paradigm stimulation. Therefore, the starting moment of each image stimulation is defined as the zero moment, and the EEG signal data is segmented and intercepted in accordance with a first preset time window ([−0.2, 0.8] seconds in this embodiment).

The baseline correction is specified as: baseline correction of the EEG signal data obtained from each image stimulation (EEG signal data segments intercepted in segments in accordance with the first preset time window) by using the EEG signal data segments of the second preset time window (in this embodiment, [−0.2, 0] seconds) as a baseline to prevent the effect of data drift.

In one embodiment, said pre-processing of the before LTT standard stimulation EEG signal data, the before LTT target stimulation EEG signal data, the after LTT standard stimulation EEG signal data and the after LTT target stimulation EEG signal data to obtain the before LTT standard stimulation event-related potential feature, the before LTT target stimulation event-related potential feature, the after LTT standard stimulation event-related potential feature and the after LTT target stimulation event-related potential features, including:

Based on the preprocessed EEG signal data, the event-related potential characteristics were obtained by averaging the EEG signal data of each trial separately at each electrode by superposition calculation.

Stacked averaging of EEG signal data is calculated to reduce noise so that event-related brain activity can be more easily observed. ERPs are characterized as time-locked and phase-locked, i.e., constant latency and constant waveform. The acquired single-trial EEG data contains event-related brain activity (i.e., ERP), as well as other non-event-related activities (e.g., skin potentials, EMG artifacts, ocular artifacts, and induced noise, etc.), which are randomly varying with high and low levels, and thus can be averaged by superimposing the EEG data from multiple segments, so that the random irrelevant signals positively and negatively cancel each other out, and finally an accurate ERP is extracted. cancel each other out, and finally extract the accurate P300 amplitude features to form the ERP waveform (also known as time-domain waveform).

In one embodiment, the event-related potentials characterizing the healthy population and the iNPH patient are expressed differently.

The manifestations of event-related potential features in healthy populations are specified as: before LTT standard stimulus event-related potential features, before LTT target stimulus event-related potential features, after LTT standard stimulus event-related potential features, and after LTT target stimulus event-related potential features reflecting no change in cognitive function in healthy populations;

The manifestations of the event-related potential features of iNPH patients were specifically: before LTT standard stimulus event-related potential features, before LTT target stimulus event-related potential features, after LTT standard stimulus event-related potential features, and after LTT target stimulus event-related potential features reflecting improved cognitive functioning in iNPH patients but not reaching the minimum level of cognitive functioning in the healthy population;

Wherein, said iNPH prediction model is trained to obtain the iNPH prediction model based on the before LTT standard stimulus event-related potential feature, the before LTT target stimulus event-related potential feature, the before LTT standard stimulus event-related potential feature, and the before LTT target stimulus event-related potential feature, as follows:

The iNPH prediction model was trained to obtain the iNPH prediction model based on the change characteristics of the standard stimulus event-related potential feature before LTT, the target stimulus event-related potential feature before LTT, the standard stimulus event-related potential feature after LTT, and the target stimulus event-related potential feature after LTT.

The main scalp distribution of the P300 amplitude feature was at the midline electrodes (Fz, Cz, and Pz), and its amplitude typically increased from the forehead to the parietal electrode site.

Referring to FIG. 4, which illustrates the results of the visual Oddball paradigm experiments in a healthy population. Target (solid line) is the ERP waveform under the target stimulus, and standard (dashed line) is the ERP waveform under the standard stimulus, and the target stimulus was able to induce a larger P300 wave amplitude compared to the standard stimulus. This is a change in the event-related potential characteristics of the healthy population, which does not need to undergo LTT, and can be performed at the same time before and after LTT of iNPH patients, the visual Oddball paradigm experiment can be performed simultaneously on the healthy population, which will find that the results of the two visual Oddball paradigm experiments of the healthy population are consistent, and there is no change in cognitive function, even if there may be a slight difference, which is a negligible change even if there may be a slight difference, it is still a negligible change.

Referring to FIG. 5, FIG. 5 illustrates the results of the visual oddball paradigm experiments in the iNPH population, FIGS. 5(a) and 5(b) show the time-domain waveforms under the visual oddball paradigm experiments before and after LTT, respectively. Target (solid line) is the ERP waveform under the target stimulus, and standard (dashed line) is the ERP waveform under the standard stimulus, and the target stimulus evoked larger P300 waveform amplitude compared with the standard stimulus, but the P300 amplitude was still lower compared to the healthy population, and the difference between the target stimulus and the standard stimulus was also smaller. FIG. 5(c) shows the comparison of the time domain waveforms under the target stimulus in the visual oddball paradigm experiments before and after LTT, target (solid line) is the ERP waveform of the target stimulus after LLTT, and standard (dashed line) is the ERP waveform of the target stimulus before LTT, and it can be seen that the P300 amplitude is larger than that of the target stimulus after LTT than before LTT. From this, it can be obtained that the P300 amplitude characteristics of iNPH patients before and after LTT changed significantly, but the effect did not reach the lowest cognitive function level of the healthy population (i.e., even though iNPH patients got cognitive improvement by performing LTT, their cognitive function level was worse than those with the lowest cognitive function level in the healthy population in any case), which can be shown that LTT improved cognitive function level of iNPH patients, but still falls short of the lowest cognitive function in the healthy population.

Therefore, an iNPH prediction model can be trained to obtain based on the EEG signal data of a healthy population and an iNPH stimulated with a visual Oddball paradigm, analyzed and learned from the changes in the P300 amplitude feature, for use in assisting physicians in diagnosing suspected iNPH patients. It should be noted that the iNPH prediction model can adopt any compliant modeling framework in the prior art, such as a binary classification model, etc.

In assisting physicians to diagnose patients with suspected iNPH, the visual Oddball paradigm experiment was first performed on the tester before the tester underwent LTT to obtain the tester's before LTT standard stimulus EEG signal data and the before LTT target stimulus EEG signal data; Then after the tester underwent LTT, the same visual Oddball paradigm experiment was performed on the test subject to obtain the test subject's after LTT standard stimulus EEG signal data and after LTT target stimulus EEG signal data; Then the before LTT standard stimulus EEG signal data, the before LTT target stimulus EEG signal data, the after LTT standard stimulus EEG signal data and the after LTT target stimulus EEG signal data were pre-processed to obtain the before LTT standard stimulus event-related potential characteristics, the before LTT target stimulus event-related potential characteristics, the after LTT standard stimulus event-related potential characteristics, and the after LTT target stimulus signal characteristics. Where the event-related potential features are P300 amplitude features; Finally, based on the before LTT standard stimulus event-related potential features, before LTT target stimulus event-related potential features, after LTT standard stimulus event-related potential features and after LTT target stimulus event-related potential features, through the iNPH prediction model obtained by the above construction method of the iNPH prediction model based on the visual Oddball paradigm, doctors can further determine the diagnosis results according to the model prediction results and the actual situation, so as to improve the accuracy of the diagnosis results.

The present invention provides an iNPH prediction method based on the visual Oddball paradigm, and designs a visual Oddball paradigm experiment that is not easily affected by subjective factors such as the patient's cultural level and life experience, and the target population passively undergoes the visual Oddball paradigm experiment before and after LTT, which effectively induces the ERP features, and can obtain more objective EEG signal data. By analyzing the characteristic changes of the event-related potential feature (P300 amplitude feature) of the EEG signal data, the degree of cognitive function improvement can be quantified, and the iNPH prediction model obtained from this training can be used to assist doctors to quickly and accurately carry out the diagnosis of iNPH, so as to enable the patients to receive timely and effective treatment, and to determine the effectiveness of the shunt surgery to improve the symptoms of iNPH, which can reduce the burden of medical treatment and bring considerable.

The following describes the construction apparatus for the iNPH prediction model based on the visual Oddball paradigm provided by the present invention, and the construction apparatus for the iNPH prediction model based on the visual Oddball paradigm described below and the method for constructing the iNPH prediction model based on the visual Oddball paradigm described above may be referred to in correspondence.

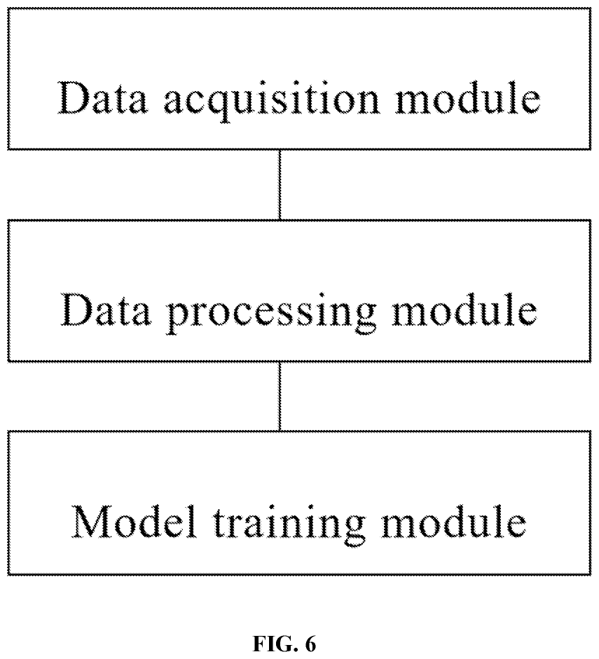

Referring to FIG. 6, the present invention provides an apparatus for constructing an iNPH predictive model based on a visual Oddball paradigm, which may include:

The data acquisition module is configured to: The target population received the standard stimulation EEG data and target stimulation EEG data obtained by visual Oddball paradigm experiment before LTT. The standard stimulation EEG data and target stimulation EEG data were obtained from the same visual Oddball paradigm experiment after LTT.

The data processing module is configured to: The data acquisition module preprocesses the standard stimulation EEG data before LTT, the target stimulation EEG data before LTT, the standard stimulation EEG data after LTT and the target stimulation EEG data after LTT. The characteristics of event-related potential to standard stimulus before LTT, event-related potential to target stimulus before LTT, event-related potential to standard stimulus after LTT and event-related potential to target stimulus after LTT were obtained, in which event-related potential features were P300 amplitude features.

The model training module is configured to train to obtain an iNPH prediction model based on the before LTT standard stimulus event related potential features, the before LTT target stimulus event related potential features, the after LTT standard stimulus event related potential features, and the after LTT target stimulus event related potential features obtained by said data processing module;

One of the target populations is iNPH patients.

The present invention also provides an iNPH prediction device based on the visual Oddball paradigm, comprising:

The data acquisition module is configured to: The standard stimulation EEG data and target stimulation EEG data obtained by visual Oddball paradigm experiment before LTT were obtained. The standard stimulation EEG data and target stimulation EEG data were obtained by the same visual Oddball paradigm experiment after LTT.

The iNPH prediction module is configured to: The iNPH prediction model is obtained by inputting the before LTT standard stimulation EEG data, before LTT target stimulation EEG data, after LTT standard stimulation EEG data and after LTT target stimulation EEG data obtained by the data acquisition module into any of the above iNPH prediction model construction methods based on visual Oddball paradigm. iNPH prediction results were obtained.

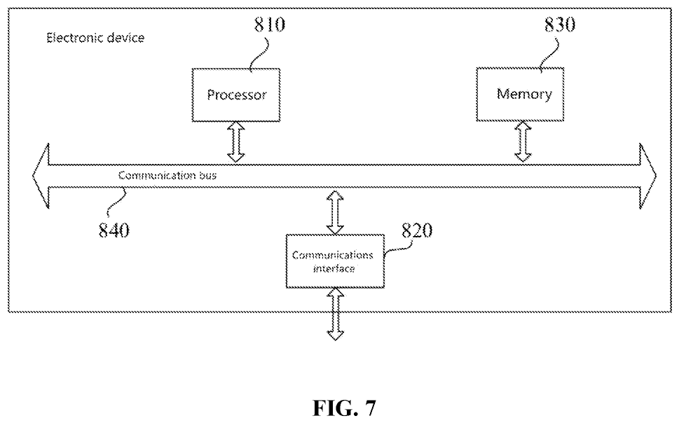

FIG. 7 exemplifies a schematic diagram of a physical structure of an electronic device, as shown in FIG. 7, which may include: a processor (processor) 810, a communication interface (communications interface) 820, a memory (memory) 830, and a communication bus 840, wherein the processor 810, the communication interface 820, the memory 830 accomplish communication with each other through the communication bus 840. The processor 810 may invoke logic instructions in the memory 830 to perform the following steps:
Obtain before LTT standard stimulus EEG signal data and before LTT target stimulus EEG signal data obtained from a visual Oddball paradigm experiment before the tester underwent LTT, and obtain after LTT standard stimulus EEG signal data and after LTT target stimulus EEG signal data obtained from the same visual Oddball paradigm experiment after the tester underwent LTT;
Pre-processing was performed on the before LTT standard stimulation EEG signal data, before LTT target stimulation EEG signal data, after LTT standard stimulation EEG signal data, and after LTT target stimulation EEG signal data;
The preprocessed before LTT standard stimulation EEG signal data, before LTT target stimulation EEG signal data, after LTT standard stimulation EEG signal data and after LTT target stimulation EEG signal data are inputted into the iNPH prediction model obtained by the method of constructing an iNPH prediction model based on the visual Oddball paradigm described in any of the foregoing claims, to obtain the prediction result of whether the tester is an iNPH patient or not;
wherein said tester is a patient with suspected iNPH, a patient with iNPH to be ruled out, or a patient with a triad of manifestations of gait disturbance, urinary incontinence, and cognitive impairment.

Furthermore, the logical instructions in the above-described memory 830 may be stored in a computer-readable storage medium when they can be realized in the form of a software function unit and sold or used as a separate product. Based on this understanding, the technical solution of the present invention essentially or contributing to the prior art or part of the technical solution may be embodied in the form of a software product that is stored in a storage medium comprising a number of instructions to cause a computer device (which may be a personal computer, a server, or a network device, etc.) to perform all or part of the steps of the method described in the various embodiments of the present invention. the method described in various embodiments of the present invention. The aforementioned storage media include: USB flash drive, portable hard disk, read-only memory (ROM, Read-Only Memory), random access memory (RAM, Random Access Memory), diskette or CD-ROM, and other media that can store program code.

The above-described embodiments of the device are merely schematic, wherein the units described as illustrated as separated components may or may not be physically separated, and the components shown as units may or may not be physical units, i.e., they may be located in a single place or they may also be distributed to a plurality of network units. Some or all of these modules may be selected to fulfill the purpose of the embodiment scheme according to actual needs. It can be understood and implemented by a person of ordinary skill in the art without creative labor.

Through the above description of the embodiments, it is clear to those skilled in the art that the embodiments can be realized with the aid of software plus the necessary general hardware platform, and of course also through hardware. Based on this understanding, the above technical solutions which essentially or rather contribute to the prior art may be embodied in the form of a software product, which may be stored in a computer-readable storage medium such as a ROM/RAM, a disk, a CD-ROM, etc., and comprises a number of instructions to enable a computer device (which may be a personal computer, a server, or a network device, etc.) to execute the respective embodiments or parts of embodiments. embodiments or certain portions of embodiments.

Finally, it should be noted that the above embodiments are only used to illustrate the technical solutions of the present invention, not to limit them; although the present invention has been described in detail with reference to the foregoing embodiments, a person of ordinary skill in the art should understand that it is still possible to make modifications to the technical solutions described in the foregoing embodiments, or to make equivalent substitutions for some of the technical features therein; and such modifications or substitutions do not take the essence of the corresponding technical solutions out of the spirit and scope of the technical solutions of the various embodiments of the present invention. These modifications or substitutions do not cause the essence of the corresponding technical solutions to depart from the spirit and scope of the technical solutions of the embodiments of the present invention.

What is claimed is:

1. A method for constructing an iNPH prediction model based on the visual Oddball paradigm, the method comprises:
performing visual Oddball paradigm experiments on a target population prior to LTT to obtain before LTT standard stimulation EEG signal data and before LTT target stimulation EEG signal data for the target population;
performing the same visual Oddball paradigm experiment on the target population after LTT to obtain after LTT standard stimulation EEG signal data and after LTT target stimulation EEG signal data for the target population;
preprocessing on the before LTT standard stimulation EEG signal data, the before LTT target stimulation EEG signal data, the after LTT standard stimulation EEG signal data and the after LTT target stimulation EEG signal data, to obtain the before LTT standard stimulation event-related potential features, the before LTT target stimulation event-related potential features, the after LTT standard stimulation event-related potential features and the after LTT target stimulation event-related potential features, wherein event-related potential features are P300 amplitude features;
training the iNPH prediction model based on the before LTT standard stimulation event-related potential characteristics, before LTT target stimulation event-related potential characteristics, after LTT standard stimulation event-related potential characteristics, and after LTT target stimulation event-related potential characteristics; and
one of the target populations is iNPH patients.

2. The method for constructing the iNPH prediction model based on the visual Oddball paradigm according to claim 1, wherein said experimenting with the visual Oddball paradigm on a target population comprises:
configuring EEG caps for the target population;
presenting a number of images to the target population at predetermined time intervals, wherein stimuli with a high probability of image occurrence are defined as standard stimuli and stimuli with a low probability of image occurrence are defined as target stimuli in the visual Oddball paradigm experiment, and asking the target population to silently memorize the number of times that the target stimuli appeared; and
presenting the images to the target population, wherein the EEG signal data of the target population is obtained through the EEG cap.

3. The method for constructing the iNPH prediction model based on the visual Oddball paradigm according to claim 2, wherein said presenting the number of images to the target population at the predetermined time interval comprises:
presenting a plurality of rounds of image stimuli to the target population, each round of image stimuli comprising a first patterned image stimulus and a second patterned image stimulus, wherein the probability of the first patterned image appearing is 80% and the first patterned image stimulus is a standard stimulus, the probability of the second patterned image appearing is 20% and the second patterned image stimulus is a target stimulus, and wherein there is a predetermined resting time between each round of image stimuli; and
presenting the first pattern image or the second pattern image to the target population each time in accordance with a predetermined maintenance duration,
wherein there is a predetermined time interval between each pattern image stimulation.

4. The method for constructing the iNPH prediction model based on the visual Oddball paradigm according to claim 3, wherein said configuring the EEG cap for the target population comprises:
equipping the target population with EEG caps, which used standard Ag/AgCl electrodes, with electrodes placed with reference to the international 10-20 system, wherein the number of channels is 32 leads; and
setting the parameters of the EEG caps to a sampling rate of 1,000 Hz and a band-pass filtering of 0.1 to 200 Hz, wherein the acquisition process is performed with the top of the head of the target population as a reference, with the forehead grounded and the impedance between the scalp and the electrodes are maintained at less than 10 KΩ.

5. The method for constructing the iNPH prediction model based on the visual Oddball paradigm according to claim 4, wherein said preprocessing comprises filtering out invalid data, filtering, downsampling, data segmentation, and baseline correction, wherein:
the filtering out invalid data comprises checking for bad lead data in the EEG signal data and filtering it out;
the filtering comprises filtering the EEG signal data using a filter with a filter range of 0.5 to 30 Hz;
the downsampling comprises downsampling the EEG signal data to 200 Hz;
the data segmentation comprises:
defining the start moment of each image stimulation as the zero moment; and
segmenting and intercepting the EEG signal data in accordance with a first predetermined time window; and
the baseline correction comprises baseline correcting the EEG signal data obtained from each image stimulation by using the EEG signal data segment of the second predetermined time window as a baseline.

6. The method for constructing the iNPH prediction model based on the visual Oddball paradigm according to claim 5, wherein said pre-processing of the before LTT standard stimulation EEG signal data, the before LTT target stimulation EEG signal data, the after LTT standard stimulation EEG signal data, and the after LTT target stimulation EEG signal data, to obtain the before LTT standard stimulus stimulation event-related potential feature, the before LTT target stimulation event-related potential features, the after LTT standard stimulation event-related potential features and the after LTT target stimulation event-related potential features, includes obtaining the event-related potential characteristics based on the preprocessed EEG signal data, by averaging the EEG signal data of each trial separately at each electrode by overlaying the calculation.

7. The method for constructing the iNPH prediction model based on the visual Oddball paradigm according to claim 1, wherein:
the presentation of the event-related potential characteristics of the target group comprises:
a standard stimulus event-related potential characteristic before LTT;
a target stimulus event-related potential characteristic before LTT;
a standard stimulus event-related potential characteristic after LTT; and
a target stimulus event-related potential features after LTT, wherein the presentation of the event-related potential characteristics of the target group is configured to reflect improvement in the cognitive function of iNPH patients without reaching the minimum cognitive function level of a healthy population; and
training said iNPH prediction model to obtain the iNPH prediction model based on the before LTT standard stimulus event-related potential feature, the before LTT target stimulus event-related potential feature, the after LTT standard stimulus event-related potential feature, and the after LTT target stimulus event-related potential feature, comprises training the iNPH prediction model to obtain the iNPH prediction model based on a change in characteristics of the standard stimulus event-related potential feature before LTT, the target stimulus event-related potential feature before LTT, the standard stimulus event-related potential feature after LTT, and the target stimulus event-related potential feature after LTT.

8. An apparatus for constructing an iNPH prediction model based on a visual Oddball paradigm, wherein the apparatus comprises:
a data acquisition module configured to receive a standard stimulation EEG data and target stimulation EEG data obtained by visual Oddball paradigm experiment before LTT on a target population wherein the standard stimulation EEG data and target stimulation EEG data are obtained from the same visual Oddball paradigm experiment after LTT;
a data processing module configured to preprocess the standard stimulation EEG data before LTT, the target stimulation EEG data before LTT, the standard stimulation EEG data after LTT and the target stimulation EEG data after LTT wherein characteristics of potential related to standard stimulus events before LTT, potential related to target stimulus events before LTT, potential related to standard stimulus events after LTT and potential related to target stimulus events after LTT are obtained, in which event-related potential features are P300 amplitude features;
a model training module configured to train the iNPH prediction model according to the potential characteristics of the standard stimulus event before LTT, the potential characteristics of the target stimulus event before LTT, the potential characteristics of the standard stimulus event after LTT, and the potential characteristics of the target stimulus event after LTT; and
one of the target populations is iNPH patients.

9. An iNPH prediction device based on the visual Oddball paradigm, wherein the device comprises:
a data acquisition module configured to obtain a standard stimulation EEG data and target stimulation EEG data by visual Oddball paradigm experiment before LTT are obtained wherein the standard stimulation EEG data and target stimulation EEG data are obtained by the same visual Oddball paradigm experiment after LTT; and
a iNPH prediction module configured to obtain the iNPH prediction mode by inputting the before LTT standard stimulation EEG data, the before LTT target stimulation EEG data, the after LTT standard stimulation EEG data and the after LTT target stimulation EEG data obtained by the data acquisition module into the construction method of the iNPH prediction model based on the visual Oddball paradigm as described in claim 1 and obtain iNPH prediction results.

10. A brain-computer interface system for predicting iNPH, wherein the system comprises:
an EEG cap for being worn on a head of a tester to obtain EEG signals from said tester;
an EEG acquisition device configured to be coupled to electrodes of said EEG cap to acquire EEG signals of said tester acquired by said EEG cap;
a computer comprising a communication module, a memory, a processor, and a computer program stored on said memory and runnable on said processor, wherein said computer is configured to communicate with said electroencephalographic acquisition device to obtain an electroencephalographic signal from said tester, and wherein said processor is configured to implement the following steps in executing said computer program:
obtain pre-drain standard stimulus EEG signal data and before LTT target stimulus EEG signal data obtained from a visual Oddball paradigm experiment before the tester underwent LTT, and obtain after LTT standard stimulus EEG signal data and after LTT target stimulus EEG signal data obtained from the same visual Oddball paradigm experiment after the tester underwent LTT; and
perform preprocessing on the before LTT standard stimulation EEG signal data, before LTT target stimulation EEG signal data, after LTT standard stimulation EEG signal data and after LTT target stimulation EEG signal data, wherein the preprocessed before LTT standard stimulation EEG signal data, the before LTT target stimulation EEG signal data, the after LTT standard stimulation EEG signal data, and the after LTT target stimulation EEG signal data are input into an iNPH prediction model obtained by the method for constructing an iNPH prediction model based on the visual Oddball paradigm as described in claim 1, and the iNPH prediction model obtained by the method of constructing an iNPH prediction model based on the visual Oddball paradigm is obtained, and whether the tester is an iNPH patient is predicted.
